(19) United States
(12) Patent Application Publication
(10) Pub. No.: US 2010/0015240 A1
(43) Pub. Date: Jan. 21, 2010

(54) PROCESS FOR PREPARING MICROPARTICLES CONTAINING BIOACTIVE PEPTIDES

(76) Inventor: Danielle Biggs, Collierville, TN (US)

Correspondence Address: Ballard Spahr LLP, SUITE 1000, 999 PEACHTREE STREET, ATLANTA, GA, 30309-3915, US

(21) Appl. No.: 12/504,078

(22) Filed: Jul. 16, 2009

Related U.S. Application Data
(60) Provisional application No. 61/081,264, filed on Jul. 16, 2008.

(51) Int. Cl. A61K 9/14; A61K 31/74; A61K 31/76; A61K 31/785; A61K 31/765; A61K 38/16; A61K 38/31; A61K 38/27; A61P 9/10

(52) U.S. Cl. 424/499; 424/78.08; 424/78.31; 424/78.37; 514/15; 514/14; 514/12; 424/501

(57) ABSTRACT

The present disclosure relates to processes for preparing microparticles comprising peptides and to microparticles prepared by such processes. Also disclosed are methods for delivering a bioactive peptide to a subject in need of treatment by the bioactive peptide.

### CROSS REFERENCE TO RELATED APPLICATIONS

This application claims the benefit of priority to U.S. Provisional Application 61/081,264, filed Jul. 16, 2008, which is incorporated by reference herein in its entirety.

### FIELD

The present disclosure relates to processes for preparing microparticles comprising peptides and to microparticles prepared by such processes. Also disclosed are methods for delivering a bioactive peptide to a subject in need of treatment by the bioactive peptide.

### BACKGROUND

Microparticles have been used to deliver a wide range of active ingredients from perfumes to pharmaceuticals. However, the ability to efficiently and effectively incorporate certain types of active ingredients into microparticles, especially amino acid-comprising compounds like peptides, can be limited by several factors. For example, the solubility of some bioactive peptides, inter alia, goserelin, leuprolide, and octreotide, is highly limited in the organic solvents typically used in the preparation of microparticles. Therefore, the loading of bioactive peptides into microparticles is limited to a relatively low level with low efficiency of loading. Further, bioactive peptides are often released quickly from microparticles (i.e., high burst). While not wishing to be bound by theory, the high burst is believed to be the result of the bioactive peptide being distributed in the microparticle as “chunks,” which is likely due to the limited solubility of peptides in the organic solvents used in the preparation of the microparticles.

There is therefore a need for processes that can overcome the low efficiency and effectiveness of current state of the art processes for incorporating bioactive peptides into microparticles. There is also a need for microparticles containing bioactive peptides that have a reduced burst. The compositions and methods address these and other needs.

### SUMMARY

In accordance with the purposes of the disclosed materials, compositions, articles, devices, and methods, as embodied and broadly described herein, the disclosed subject matter, in one aspect, relates to compounds and compositions, and to methods for providing and using such compounds and compositions. Also, disclosed herein is an oil-in-water emulsion process for preparing microparticles that can deliver high levels of peptides wherein propylene glycol is used to dissolve the peptide prior to combination with a solution comprising the wall forming polymer excipient. The microparticles formed by the disclosed process have high encapsulation efficiencies as well as improved drug release characteristics (including, for example, reduced initial burst of drug). The process can be adapted to a water-in-oil-in-water double emulsion process; further, the process can be adapted to water-in-oil and oil-in-water-in-oil processes as well. The present disclosure further relates to the use of the microparticles prepared by the disclosed process to treat one or more diseases or medical conditions treatable by bioactive peptides.

Additional advantages will be set forth in part in the description that follows, and in part will be obvious from the description, or can be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

### DETAILED DESCRIPTION

Before the present processes, homopolymers, copolymers, polymer admixtures, compounds, and/or compositions are disclosed and described, it is to be understood that the aspects described herein are not limited to specific processes, compounds, synthetic methods, or uses as such can, of course, vary. It is also to be understood that the terminology used herein is for the purpose of describing particular aspects only and, unless specifically defined herein, is not intended to be limiting.

Also, throughout this specification, various publications are referenced. The disclosures of these publications in their entireties are hereby incorporated by reference into this application in order to more fully describe the state of the art to which the disclosed matter pertains. The references disclosed are also individually and specifically incorporated by reference herein for the material contained in them that is discussed in the sentence in which the reference is relied upon.

### Definitions

In this specification and in the claims that follow, reference will be made to a number of terms, which shall be defined to have the following meanings:

Throughout the description and claims of this specification the word “comprise” and other forms of the word, such as “comprising” and “comprises,” means including but not limited to, and is not intended to exclude, for example, other additives, components, integers, or steps.

It must be noted that, as used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

“Optional” or “optionally” means that the subsequently described event or circumstance can or cannot occur, and that the description includes instances where the event or circumstance occurs and instances where it does not.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

A weight percent of a component, unless specifically stated to the contrary, is based on the total weight of the formulation or composition in which the component is included.

By “contacting” is meant the physical contact of at least one substance to another substance.

By “combining” is meant the physical admixing of two or more polymers, ingredients, phases, solutions, and the like in any order.

By “sufficient amount” and “sufficient time” means an amount and time needed to achieve the desired result or results, e.g., dissolve a portion of the polymer.

“Polymer excipient” or “polymer” as used herein refers to homopolymer or copolymer or blends comprising homopolymers and/or copolymers and combinations or blends thereof that are used as the microparticle wall forming or matrix materials. This term should be distinguished from the term “excipient” as defined herein below.

“Molecular weight” as used herein, unless otherwise specified, refers generally to the relative average molecular weight of the bulk polymer. In practice, molecular weight can be estimated or characterized in various ways including gel permeation chromatography (GPC) or capillary viscometry. GPC molecular weights are reported as the weight-average molecular weight (Mw) or as the number-average molecular weight (Mn). Capillary viscometry provides estimates of molecular weight as the Inherent Viscosity (IV) determined from a dilute polymer solution using a particular set of concentration, temperature, and solvent conditions. Unless otherwise specified, IV measurements are made at 30° C. on solutions prepared in chloroform at a polymer concentration of 0.5 g/dL.

“Controlled release” as used herein means the use of a material to regulate the release of another substance.

“Peptide” is used herein to include any poly amino acid having from about 5 to about 200 amino acids residues, for example, from about 5 to about 100 amino acids residues, or from about 5 to about 50 amino acids residues. “Peptides” can be a single chain having any form, for example, a linear peptide, a branched peptide, or a cyclic peptide. The term “peptide” disclosed herein can be naturally occurring or synthetic.

“Excipient” is used herein to include any other compound or additive that can be contained in or on the microparticle that is not a therapeutically or biologically active compound. As such, an excipient should be pharmaceutically or biologically acceptable or relevant (for example, an excipient should generally be non-toxic to the subject). “Excipient” includes a single such compound and is also intended to include a plurality of excipients. This term should be distinguished from the term “polymer excipients” as defined above.

“Agent” is used herein to refer generally to compounds that are contained in or on a microparticle composition. Agent can include a bioactive agent or an excipient. “Agent” includes a single such compound and is also intended to include a plurality of such compounds.

The term “microparticle” is used herein to include nanoparticles, microspheres, nanospheres, microcapsules, nanocapsules, and particles, in general. As such, the term microparticle refers to particles having a variety of internal structure and organizations including homogeneous matrices such as microspheres (and nanospheres) or heterogeneous core-shell matrices (such as microcapsules and nanocapsules), porous particles, multi-layer particles, among others. The term “microparticle” refers generally to particles that have sizes in the range of about 10 nanometers (nm) to about 2 mm (millimeters).

Unless stated to the contrary, a formula with chemical bonds shown only as solid lines and not as wedged or dashed lines contemplates each possible isomer, e.g., each enantiomer and diastereomer, and a mixture of isomers, such as a racemic or scalemic mixtures.

Enantiomeric species may exist in different isomeric or enantiomeric forms. Unless otherwise specified, enantiomeric species discussed herein without reference to their isomeric form shall include all various isomeric forms as well as racemic and scalemic mixtures of isomeric forms. For example, reference to lactic acid shall herein include L-lactic acid, D-lactic acid, and mixtures of the L- and D-isomers of lactic acid; reference to lactide shall herein include L-lactide, D-lactide, and DL-lactide (where DL-lactide refers to mixtures of the L- and D-isomers of lactide); similarly, reference to poly(lactide) shall herein include poly(L-lactide), poly(D-lactide) and poly(DL-lactide); similarly, reference to poly(lactide-co-glycolide) will herein include poly(L-lactide-co-glycolide), poly(D-lactide-co-glycolide), and poly(DL-lactide-co-glycolide); and so on.

### Methods

The disclosed process provides several unmet needs. For example, it has been discovered that peptides formulated into microparticles under the conditions of the disclosed process have improved drug release properties. The initial burst or release of peptide is reduced thereby providing a method of controllably releasing a peptide over a pre-determined amount of time. This leveling of release rate allows the formulator to produce microparticles that can be formulated with time and dose sensitive peptides.

In addition, because the peptide can be fully dissolved prior to entering the encapsulation process, the solutions can be sterile-filtered thereby facilitating the delivery of a pharmaceutically acceptable ingredient. It is well know that preparing a bulk drug powder and then sterilizing it before microencapsulation processing can be accomplished. But it is a complex and costly process to implement and can result in the degradation of peptides that comprise the microparticle. The disclosed process removes this processing problem.

In addition, when dry powder peptides are added to a solution of a wall forming polymer excipient, these peptides can agglomerate or not disperse easily leading to a non-homogeneous system or requiring longer processing and exposure times of the drug in the polymeric solution. Further, the disclosed process allows for dissolution of the peptide under controlled conditions that allows for aseptic formulation. In addition, the homogeneous distribution of the peptide in the initial phase does not require the peptide to be in any particular form in the dispersed phase. This is especially true if during processing the peptide is prone to precipitate from solution. As such, the precipitation of the peptide will be from a homogeneous solution and therefore still provide the formulator with controlled, uniform loading.

Disclosed herein is a process for preparing peptide-containing microparticles, the process comprising:
a) providing one or more peptides;
b) dissolving the one or more peptides in a solution comprising propylene glycol to form a peptide solution;
c) providing a solution comprising a polymer excipient dissolved or dispersed therein;
d) combining the peptide solution from (b) with the polymer excipient solution from (c) to form a dispersed phase;
e) providing a continuous phase comprising water;
f) combining the dispersed phase and the continuous phase to form an emulsion;
g) combining the emulsion formed in step (f) with an extraction phase comprising water; and
h) forming microparticles.

Emulsion-based processes are well known and involve the preparation, by one means or another, of a liquid-liquid dispersion. In the disclosed process, this dispersion comprises the solution from step (b) and the solution from step (c). The solution from step (b) comprises a peptide and propylene glycol. As disclosed herein, it has been found that propylene glycol has better peptide-solubility properties when compared to closely-related glycerol and the low molecular weight liquid polyol PEG-400. The solution from step (c) comprises a homopolymer, copolymer, or a mixture thereof that will form the matrix of the microparticle. The term “matrix forming polymer” is used throughout the specification to describe the polymer or combination of polymers that comprise the solution or dispersion of step (c) or the second phase of the dispersed phase. This phase formed in step (d) is known as the “dispersed phase” or “dispersed phase solution,” because it is discontinuous in the second phase of step (e), known as the “continuous phase” or “continuous phase solution.” Once the dispersed phase and the continuous phase are combined or contacted together in step (f) an emulsion forms. Once formed, this emulsion is then further diluted with an additional solvent or solution, known as the “extraction phase” (EP) or “extraction solution.”

The dispersed phase formed in step (d) of the disclosed process comprises a matrix-forming polymer as further described herein. Upon addition and dispersion of the peptide solution from step (b) into the polymer solution of step (c), the result can be a homogeneous dispersed phase solution when the peptide remains dissolved in the final dispersed phase system. Alternatively, the resulting dispersed phase system can be a suspension comprising both dissolved peptide and dispersed peptide, the ratio of which is based on the solubility of the peptide in the final DP system. Alternatively, the resulting dispersed phase system can be an emulsion of two or more immiscible phases. In instances where peptide can precipitate out of solution during step (d), mixing or agitation or turbulence or energy by any suitable means can be used during the precipitation process in order to control or reduce particle size of the peptide during precipitation. Further, excipients such as salts, counter-ions, or solvents may be added to either or both the peptide solution from step (b) or the polymer solution of step (c) in order to facilitate reprecipitation to a particular solid-state form of the drug such as one or more salt forms of the peptide, solvate forms of the peptide, polymorphic forms of the peptide, and so on.

When the dispersed phase/continuous phase emulsion is combined with the extraction phase, the resulting loss of solvent or solvents from the dispersed phase into the extraction phase causes the discontinuous droplets of the dispersed phase to harden into polymer-rich microparticles that comprise the peptide. The disclosed process is further described in detail herein below.

### Peptides

As used herein the term “peptide” refers to a linear, branched or cyclic polyamino acid chain comprising from 5 to about 200, from about 5 to about 100, or from about 5 to about 50 amino acid residues. For example, the peptides can have a molecular weight of from about 500 Daltons to about 22,000 Daltons, from about 500 Daltons to about 10,000 Daltons, or from about 500 Daltons to about 5000 Daltons. The amino acids can be the common naturally occurring L-amino acids found in most living cells, for example, alanine, arginine, asparagine, aspartic acid, cysteine, glutamic acid, glutamine, glycine, histidine, isoleucine, leucine, lysine, methionine, phenylalanine, praline, serine, threonine, tryptophan, tyrosine, and valine. Or the amino acid can be the D-configuration or can be racemic or comprise an excess of either the L- or D-configuration. In addition, non-naturally occurring amino acids can comprise the peptides, for example, β-alanine, homoserine, homoleucine, naphthylalanine, aziridine-2-carboxylic acid, azetidine-2-carboxylic acid, piperidine-2-carboxylic acid, and piperidine-3-carboxylic. The term “peptide” is used herein to include naturally-occurring or synthetic peptides, e.g., bioactive peptides, as well as capped, protected, or modified analogs of peptides.

One category of the disclosed peptides relates to naturally occurring bio-active peptides. Non-limiting examples of bio-active naturally occurring peptides includes oxytocin, somatostatin, angiotensin, bradykinin, arginine vasopressin, adrenocorticotropic hormone, and glucagon-like peptides.

Another category of the disclosed peptides relates to synthetic or non-naturally occurring bio-active peptides. Non-limiting examples of bio-active non-naturally occurring peptides includes goserelin, leuprolide, GLP-1 peptide analogs, GLP-2 peptide analogs, and octreotide.

Peptides included in the present invention include any bioactive peptides, whether synthetic or naturally occurring, including antithrombotic peptides, antihypertensive peptides, opioid peptides, neuroactive peptides, CNS-active peptides, immunomodulating peptides, antimicrobial peptides, caseinophosphopeptides, glycomacropeptides, metabolic peptides, antimetabolic peptides, inflammatory peptides, anti-inflammatory peptides, renally-active peptides, cardio-active peptides, gastrointestinal peptides, chemotherapeutic peptides, hematopoietic peptides, growth peptides, growth-factor peptides, inhibitory peptides, hormonally-active peptides, as well as any bioactive peptides useful in therapeutic areas cited in Goodman & Gilman's The Pharmacological Basis of Therapeutics (McGraw Hill). Non-limiting examples of bioactive peptides and classes of bioactive peptides include those cited in the Handbook of Biologically Active Peptides, A. J. Kastin (Editor), Academic Press (Elsevier), Burlington, Mass., 2006.

### Polymer Excipients

The disclosed microparticles comprise one or more wall forming polymer excipients. The polymer excipients can have an average molecular weight from about 1,000 Daltons to about 2,000,000 Daltons. Molecular weights, for example, can be determined by gel permeation chromatography (GPC) in chloroform against commercial polystyrene standards. In one embodiment, the polymer excipient has an average molecular weight of from about 2,000 Daltons to about 200,000 Daltons. In a further embodiment, the polymer excipient has an average molecular weight of from about 4,000 Daltons to about 100,000 Daltons. In another embodiment, the polymer excipient has an average molecular weight of from about 5,000 Daltons to about 50,000 Daltons. In a yet further embodiment, the polymer excipient has an average molecular weight of from about 1,000 Daltons to about 10,000 Daltons. In a still further embodiment, the polymer excipient has an average molecular weight of from about 5,000 Daltons to about 20,000 Daltons. In a still yet further embodiment, the polymer excipient has an average molecular weight of from about 8,000 Daltons to about 12,000 Daltons.

The polymer average molecular weights can be obtained be Gel Permeation Chromatography (GPC), for example, as described by L. H. Sperling of the Center for Polymer Science and Engineering & Polymer Interfaces Center, Materials Research Center, Department of Chemical Engineering and Materials Science and Engineering Department, Lehigh University, 5 E. Packer Ave., Bethlehem, Pa. 18015-3194, as first described in: ACS Division of Polymeric Materials: Science and Engineering (PMSE), 81:569 (1999).

Alternatively the molecular weights can be described by their measured Inherent Viscosity (IV) as determined by capillary viscometry using a specified temperature, concentration, and solvent. Molecular weights of the polymers or copolymers described herein can be about 0.05 dL/g to about 2.0 dL/g wherein dL is deciliter when measured, for example, at 30° C. in chloroform solutions having a polymer concentration of 0.5% (w/v). In another embodiment the inherent viscosity can be from about 0.05 dL/g to about 1.2 dL/g. In a further embodiment the inherent viscosity can be form about 0.1 dL/g to about 1.0 dL/g. A yet further embodiment of the polymers and copolymers of the present disclosure can have an inherent viscosity of from about 0.1 dL/g to about 0.8 dL/g. And yet another embodiment of the polymers and copolymers of the present disclosure can have an inherent viscosity of from about 0.05 dL/g to about 0.5 dL/g. Alternatively, the formulator can express the inherent viscosity in cm³/g if convenient.

One category of the disclosed polymer excipients relates to homopolymers or copolymers comprising lactide, glycolide, a hydroxy acid other than lactide or glycolide, and mixtures or blends thereof. Non-limiting examples of this category of polymer excipients include polymers chosen from:
i) poly(lactide);
ii) poly(glycolide);
iii) poly(caprolactone);
iv) poly(valerolactone);
v) poly(hydroxybutyrate);
vi) poly(lactide-co-glycolide);
vii) poly(lactide-co-caprolactone);
viii) poly(lactide-co-valerolactone);
ix) poly(glycolide-co-caprolactone);
x) poly(glycolide-co-valerolactone);
xi) poly(lactide-co-glycolide-co-caprolactone); and
xii) poly(lactide-co-glycolide-co-valerolactone).

One embodiment of this category relates to microparticles comprising poly(lactide), PLA. The poly(lactide) can have an average molecular weight of from 1,000 Daltons to about 2,000,000 Daltons. One iteration of microparticles formed from poly(lactide) polymer excipients are microparticles comprising poly(lactide) having an average molecular weight of from 1,000 Daltons to 60,000 Daltons. Another iteration of microparticles are microparticles comprising poly(lactide) having an average molecular weight of from 10,000 Daltons to 80,000 Daltons. A further iteration of microparticles are microparticles comprising poly(lactide) having an average molecular weight of from 1,000 Daltons to 15,000 Daltons. Poly(lactide) is available from Brookwood Pharmaceuticals (Birmingham, Ala.).

Another embodiment of this category relates to microparticles comprising poly(lactide-co-glycolide). The poly(lactide-co-glycolide) can have an average molecular weight of from 1,000 Daltons to about 2,000,000 Daltons. One iteration of microparticles formed from poly(lactide-co-glycolide) are microparticles comprising poly(lactide-co-glycolide) having an average molecular weight of from 1,000 Daltons to 60,000 Daltons. Another iteration of microparticles are microparticles comprising poly(lactide-co-glycolide) having an average molecular weight of from 10,000 Daltons to 80,000 Daltons. A further iteration of microparticles are microparticles comprising poly(lactide-co-glycolide) having an average molecular weight of from 2,000 Daltons to 15,000 Daltons. Poly(lactide-co-glycolide) is available from Brookwood Pharmaceuticals (Birmingham, Ala.). The poly(lactide-co-glycolide) can have a ratio of lactide to glycolide of from about 40 lactide units to about 60 glycolide units (40:60) to about 99 lactide units to about 1 glycolide unit (99:1). Non-limiting examples of poly(lactide-co-glycolide) suitable as wall forming polymer excipients include polymers having a ratio of lactide to glycolide of 1:1 (50:50), 1.4:1 (58:42), and 1.8:1 (64:36).

A further embodiment of this category relates to microparticles comprising poly(lactide-co-caprolactone). The poly(lactide-co-caprolactone) can have an average molecular weight of from 1,000 Daltons to about 2,000,000 Daltons. One iteration of microparticles formed from poly(lactide-co-caprolactone) are microparticles comprising poly(lactide-co-caprolactone) having an average molecular weight of from 1,000 Daltons to 60,000 Daltons. Another iteration of microparticles are microparticles comprising poly(lactide-co-caprolactone) having an average molecular weight of from 10,000 Daltons to 80,000 Daltons. A further iteration of microparticles are microparticles comprising poly(lactide-co-caprolactone) having an average molecular weight of from 2,000 Daltons to 15,000 Daltons. Poly(lactide-co-caprolactone) is available from Brookwood Pharmaceuticals (Birmingham, Ala.). The poly(lactide-co-caprolactone) can have a ratio of lactide to glycolide of from about 1 lactide unit to about 99 glycolide units (1:99) to about 99 lactide units to about 1 glycolide unit (99:1).

Another category of the disclosed polymer excipients relates block copolymers comprising homopolymers or copolymers comprising lactide, glycolide, a hydroxy acid other than lactide or glycolide, and mixtures thereof and homopolymers or copolymers of polyalkylene glycols. Non-limiting examples of this category of polymer excipients include polymers chosen from, or polymer mixtures or blends comprising:
i) poly(lactide)-co-(polyalkylene oxide);
ii) poly(lactide-co-glycolide)-co-(polyalkylene oxide);
iii) poly(lactide-co-caprolactone)-b-(polyalkylene oxide); and
iv) poly(lactide-co-glycolide-co-caprolactone)-b-(polyalkylene oxide).

One embodiment of this category relates to microparticles comprising poly(lactide)-co-(polyalkylene oxide). One iteration of this embodiment relates to microparticles comprising poly(lactide)-co-(polyethylene oxide) as the wall forming polymer excipient. Another iteration relates to microparticles comprising poly(lactide)-co-(polypropylene oxide) as the wall forming polymer excipient. A further iteration relates to microparticles comprising poly(lactide)-co-(polyethylene oxide-co-polypropylene oxide) as the wall forming polymer excipient.

Another embodiment of this category relates to microparticles comprising poly(lactide-co-glycolide)-co-(polyalkylene oxide). One iteration of this embodiment relates to microparticles comprising poly(lactide-co-glycolide)-co-(polyethylene oxide) as the wall forming polymer excipient. Another iteration relates to microparticles comprising poly(lactide-co-glycolide)-co-(polypropylene oxide) as the wall forming polymer excipient. A further iteration relates to microparticles comprising poly(lactide-co-glycolide)-co-(polyethylene oxide-co-polypropylene oxide) as the wall forming polymer excipient.

A further embodiment of this category relates to microparticles comprising poly(lactide-co-caprolactone)-co-(polyalkylene oxide). One iteration of this embodiment relates to microparticles comprising poly(lactide-co-caprolactone)-co-(polyethylene oxide) as the wall forming polymer excipient. Another iteration relates to microparticles comprising poly(lactide-co-caprolactone)-co-(polypropylene oxide) as the wall forming polymer excipient. A further iteration relates to microparticles comprising poly(lactide-co-caprolactone)-co-(polyethylene oxide-co-polypropylene oxide) as the wall forming polymer excipient.

A yet further embodiment of this category relates to microparticles comprising poly(lactide-co-glycolide-co-caprolactone)-co-(polyalkylene oxide). One iteration of this embodiment relates to microparticles comprising poly(lactide-co-glycolide-co-caprolactone)-co-(polyethylene oxide) as the wall forming polymer excipient. Another iteration relates to microparticles comprising poly(lactide-co-glycolide-co-caprolactone)-co-(polypropylene oxide) as the wall forming polymer excipient. A further iteration relates to microparticles comprising poly(lactide-co-glycolide-co-caprolactone)-co-(polyethylene oxide-co-polypropylene oxide) as the wall forming polymer excipient.

The wall forming polymer excipients of this category can have an average molecular weight of from 1,000 Daltons to about 2,000,000 Daltons. One iteration of polymers according to this category relates to polymers having an average molecular weight of from 1,000 Daltons to 60,000 Daltons. Another iteration of polymers according to this category relates to polymers having an average molecular weight of from 10,000 Daltons to 80,000 Daltons. A further iteration of polymers according to this category relates to polymers having an average molecular weight of from 1,000 Daltons to 30,000 Daltons.

A further category of the disclosed polymer excipients relates block copolymers comprising homopolymers or copolymers comprising lactide, glycolide, a hydroxy acid other than lactide or glycolide, and mixtures thereof and homopolymers or copolymers of polyalkylene glycols. Non-limiting examples of this category of polymer excipients include polymers chosen from, or polymer mixtures or blends comprising:
i) poly(lactide)-co-poly(vinylpyrrolidone);
ii) poly(lactide-co-glycolide)-co-poly(vinylpyrrolidone);
iii) poly(lactide-co-caprolactone)-b-poly(vinylpyrrolidone); and
iv) poly(lactide-co-glycolide-co-caprolactone)-b-poly(vinylpyrrolidone).

One embodiment of this category relates to microparticles comprising poly(lactide)-co-poly(vinylpyrrolidone) as the wall forming polymer excipient. Another embodiment of this category relates to microparticles comprising poly(lactide-co-glycolide)-co-poly(vinylpyrrolidone) as the wall forming polymer excipient. A further embodiment of this category relates to microparticles comprising poly(lactide-co-caprolactone)-co-poly(vinylpyrrolidone) as the wall forming polymer excipient. A yet further embodiment of this category relates to microparticles comprising poly(lactide-co-glycolide-co-caprolactone)-co-poly(vinylpyrrolidone) as the wall forming polymer excipient. The polymer excipients of this category can be prepared according to the procedure disclosed in U.S. Pat. No. 7,262,253 the entirety of which is included herein by reference.

The wall forming polymer excipients of this category can have an average molecular weight of from 1,000 Daltons to about 2,000,000 Daltons. One iteration of polymers according to this category relates to polymers having an average molecular weight of from 1,000 Daltons to 60,000 Daltons. Another iteration of polymers according to this category relates to polymers having an average molecular weight of from 10,000 Daltons to 80,000 Daltons. A further iteration of polymers according to this category relates to polymers having an average molecular weight of from 1,000 Daltons to 30,000 Daltons.

As such, the wall forming polymer excipients suitable for use in the disclosed process can be a homopolymer, copolymer, or block copolymer comprising:
i) polyesters;
ii) polyanhydrides;
iii) polyorthoesters;
iv) polyphosphazenes;
v) polyphosphates;
vi) polyphosphoesters;
vii) polydioxanones;
viii) polyphosphonates;
ix) polyhydroxyalkanoates;
x) polycarbonates;
xi) polyalkylcarbonates;
xii) polyorthocarbonates;
xiii) polyesteramides;
xiv) polyamides;
xv) polyamines;
xvi) polypeptides;
xvii) polyurethanes;
xviii) polyetheresters;
xix) polyalkylene glycols;
xx) polyalkylene oxides;
xxi) polysaccharides;
xxii) polyvinyl pyrrolidones or
xxiii) combinations or blends thereof.

### Excipients.

Microparticles of the present invention may comprise other additives or agents (excipients) in addition to the polymer or the bioactive agent. These may be incorporated in either or both step (b) or in step (c) of the process of the present invention. Excipients may include polymeric additives, salts, counter-ions, antioxidants, free-radical scavengers, preservatives, sugars, polysaccharides, and so on. These excipients may be present as processing aids, stabilizing agents for processing steps, they may be added to affect properties of the final microparticle product, they may be added to affect the performance of the final microparticle product, further, they may be present to affect the solid-state attributes of the peptide during or after processing.

### Exemplary Processes

Step (a)

Step (a) comprises providing a peptide. The peptide can be either naturally occurring, non-naturally occurring (synthetic), or the peptide can be a modified naturally occurring peptide that comprises one or more conservative substitutions wherein the conservative substitutions can be made by an organism or can be made by manipulation of the gene sequence of the naturally occurring corresponding peptide. In addition, naturally occurring or synthetic peptides can be modified to have an additional sequence of amino acids at the N-terminus or at the C-terminus. The modifications can be made to increase or decrease the activity of the peptide or the modification can be made to improve the formulatability of the peptide either in the disclosed process or to enhance the shelf life, for example, thermal stability of the peptide. In one embodiment, the disclosed peptides have a molecular weight of from about 300 Daltons to about 5,000 Daltons.

Step (b)

Step (b) relates to dissolving one or more peptides in propylene glycol to form a peptide solution. The peptides are soluble in propylene glycol in an amount of least about 1 mg/mL (or at least about 1 mg/gram using a propylene glycol density value of approximately 1.036 g/mL). Preferably, peptides are soluble in propylene glycol in an amount of at least about 10 mg/mL. The peptide solution can comprise from about 0.1% to about 99.9% by weight of propylene glycol. In one embodiment, the peptide solution can comprise from about 50% to about 99.9% by weight of propylene glycol. In another embodiment, the peptide solution can comprise from about 80% to about 99% by weight of propylene glycol. In a further embodiment, the peptide solution can comprise from about 60% to about 80% by weight of propylene glycol. In a yet further embodiment, the peptide solution can comprise from about 10% to about 60% by weight of propylene glycol. In a still further embodiment, the peptide solution can comprise from about 1% to about 10% by weight of propylene glycol. In a yet another embodiment, the peptide solution can comprise from about 25% to about 50% by weight of propylene glycol. In a still another embodiment, the peptide solution can comprise from about 10% to about 90% by weight of propylene glycol.

In one aspect, the bioactive peptide having a solubility in propylene glycol in an amount of at least 1 mg/mL can be present in the peptide solution of step (b) at a concentration that is below its solubility limit in propylene glycol. In such a case the peptide solution of step (b) would be a homogeneous solution where the peptide is dissolved in the peptide solution. In another aspect, the bioactive peptide may be present in the peptide solution of step (b) at a concentration this is approximately at its solubility limit. In still another aspect, the bioactive peptide may be present in the peptide solution of step (b) at a concentration that is above its solubility limit in the peptide solution. In this case, the peptide solution would effectively be a suspension comprising dissolved peptide in the solvent plus additional suspended drug dispersed in the peptide solution.

The peptide solution can further comprise one or more organic solvents, or alternatively, the peptide solution can comprise water. The organic solvent can be chosen from a C₁-C₁₂ alcohol, inter alia, methanol, ethanol, n-propanol, iso-propanol, n-butanol, sec-butanol, tert-butanol, pentanol, hexanol, and benzyl alcohol; C₄-C₁₀ ether, inter alia, diethyl ether, diphenyl ether, methyl butyl ether, methyl tert-butyl ether, tetrahydrofuran, pyran, 1,2-dimethoxyethane (glyme), bis(2-methoxyethyl)ether (diglyme), and dioxane; C₃-C₁₂ ester, inter alia, methyl acetate, ethyl acetate, propyl acetate, methyl propionate, ethyl propionate, methyl lactate, and ethyl lactate; C₂-C₁₀ nitrile, inter alia, acetonitrile and propionitrile; C₃-C₁₂ ketone, inter alia, acetone, butanone, 2-pentanone, 3-pentanone, 2-hexanone, 3-hexanone, and acetophenone; substituted or unsubstituted benzene, inter alia, benzene, toluene, xylene (ortho, meta, para, or mixtures thereof), chlorobenzene, and nitrobenzene; C₅-C₂₀ hydrocarbon, inter alia, n-pentane, iso-pentane, n-hexane, n-octane, and iso-octane; C₁-C₁₂ haloalkane, inter alia, methylene chloride, chloroform, carbon tetrachloride, 1,2 dichloroethane, 1,1,1-trichloroethane, and 1,1,1,2-tetrafluroethane; C₂-C₁₂ nitroalkane, or other water soluble organic solvent, inter alia, dimethyl sulfoxide, dimethylformamide, diethylformamide, dimethylacetamide, and diethyl-acetamide.

The peptide solution can comprise other excipients including the following non-limiting examples: polymeric additives, salts, counter-ions, antioxidants, free-radical scavengers, preservatives, sugars, polysaccharides, any combinations thereof. These excipients may be present as processing aids, stabilizing agents for processing steps, they may be added to affect properties of the final microparticle product, they may be added to affect the performance of the final microparticle product, further, they may be present to affect the solid-state attributes of the peptide during or after processing.

The peptide solution can comprise from about 0.1% to about 99.9% by weight of one or more peptides. In one embodiment, the peptide solution can comprise from about 0.1% to about 99% by weight of one or more peptides. In another embodiment, the peptide solution can comprise from about 0.1% to about 70% by weight of one or more peptides. In a further embodiment, the peptide solution can comprise from about 0.1% to about 50% by weight of one or more peptides. In a yet further embodiment, the peptide solution can comprise from about 0.1% to about 30% by weight of one or more peptides. In another embodiment, the peptide solution can comprise from about 50-90% by weight of one or more peptides. In a further embodiment, the peptide solution can comprise from about 40-80% by weight of one or more peptides. In still another embodiment, the peptide solution can comprise from about 30-60% by weight of one or more peptides. In yet another embodiment, the peptide solution can comprise from about 10-30% by weight of one or more peptides. In a still further embodiment, the peptide solution can comprise from about 1% to about 10% by weight of one or more peptides. In a yet another embodiment, the peptide solution can comprise from about 2% to about 10% by weight of one or more peptides. In a still another embodiment, the peptide solution can comprise from about 2% to about 5% by weight of one or more peptides.

One embodiment of the peptide solution comprises (i) from about 1% to about 99% by weight of a bioactive peptide; and (ii) from about 1% to about 99% by weight of propylene glycol.

Another embodiment ofthe peptide solution comprises (i) from about 10% to about 70% by weight of a bioactive peptide; and (ii) from about 30% to about 90% by weight of propylene glycol.

A further embodiment of the peptide solution comprises (i) from about 1% to about 50% by weight of a bioactive peptide; and (ii) from about 50% to about 99% by weight of propylene glycol.

A yet further embodiment of the peptide solution comprises (i) from about 10% to about 70% by weight of a bioactive peptide; (ii) from about 30% to about 90% by weight of propylene glycol; and (iii) one or more organic solvents.

A still further embodiment of the peptide solution comprises (i) from about 10% to about 70% by weight of a bioactive peptide; (ii) from about 30% to about 90% by weight of propylene glycol; and (iii) one or more organic solvents.

A yet still further embodiment of the peptide solution comprises (i) from about 10% to about 70% by weight of a naturally occurring bioactive peptide; (ii) from about 30% to about 90% by weight of propylene glycol; and (iii) ethyl acetate, methylene chloride, or a mixture thereof.

Step (c)

Step (c) comprises providing a solution comprising a polymer excipient dissolved or dispersed therein. The polymer excipient is a wall forming polymer that will form the matrix of the microparticle. As such, the polymer can be any homopolymer, copolymer, or block copolymer or mixtures or blends thereof as described herein above that is compatible with the disclosed process and is capable of forming microparticles comprising one or more peptides.

The solvents used to disperse or dissolve the polymer excipient to form the solution of step (c) can be any solvent, including water, that is compatible with the disclosed process. Non-limiting examples of an organic solvent suitable for use in step (c) includes solvents chosen from a C₁-C₁₂ alcohol, inter alia, methanol, ethanol, n-propanol, iso-propanol, n-butanol, sec-butanol, tert-butanol, pentanol, hexanol, and benzyl alcohol; C₄-C₁₀ ether, inter alia, diethyl ether, diphenyl ether, methyl butyl ether, methyl tert-butyl ether, tetrahydrofuran, pyran, 1,2-dimethoxyethane (glyme), bis(2-methoxyethyl)ether (diglyme), and dioxane; C₃-C₁₂ ester, inter alia, methyl acetate, ethyl acetate, propyl acetate, methyl propionate, ethyl propionate, methyl lactate, and ethyl lactate; C₂-C₁₀ nitrile, inter alia, acetonitrile and propionitrile; C₃-C₁₂ ketone, inter alia, acetone, butanone, 2-pentanone, 3-pentanone, 2-hexanone, 3-hexanone, and acetophenone; substituted or unsubstituted benzene, inter alia, benzene, toluene, xylene (ortho, meta, para, or mixtures thereof), chlorobenzene, and nitrobenzene; C₅-C₂₀ hydrocarbon, inter alia, n-pentane, iso-pentane, n-hexane, n-octane, and iso-octane; C₁-C₁₂ haloalkane, inter alia, methylene chloride, chloroform, carbon tetrachloride, 1,2 dichloroethane, 1,1,1-trichloroethane, and 1,1,1,2-tetrafluroethane; C₂-C₁₂ nitroalkane, or other water soluble organic solvent, inter alia, dimethyl sulfoxide, dimethylformamide, diethylformamide, dimethylacetamide, and diethyl-acetamide. Non-limiting examples of preferred solvents include methylene chloride and ethyl acetate.

The amount of polymer excipient dissolved or dispersed in the solvent of step (c) will depend upon many factors, for example, the amount of polymer necessary to form microparticles of a chosen diameter range, the solubility of a polymer excipient in a solvent or solvent combination, and the like.

In another aspect of the disclosed process, the solution formed in step (c) can comprise one or more excipients that can be added directly into the polymer excipient solution, alternatively, the excipients can first be dissolved or dispersed in a solvent which is then added into the polymer excipient solution. The polymer solution formed in step (c) can comprise other excipients including the following non-limiting examples: polymeric additives, salts, counter-ions, antioxidants, free-radical scavengers, preservatives, sugars, polysaccharides, any combinations thereof. Other examples of excipients that can be added to the polymer excipient solution include an adhesive, a pesticide, a fragrance, an antifoulant, a dye, a salt, an oil, an ink, a cosmetic, a catalyst, a detergent, a curing agent, a flavor, a fuel, a herbicide, a metal, a paint, a photographic agent, a biocide, a pigment, a plasticizer, a propellant, a stabilizer, a polymer additive, any combination thereof.

Step (d)

Step (d) relates to combining the peptide solution from (b) with the polymer excipient solution from (c) to form a dispersed phase. Upon addition and dispersion of the peptide solution from step (b) into the polymer solution of step (c), the result can be a homogeneous dispersed phase solution when the peptide remains dissolved in the final dispersed phase system. Alternatively, the resulting dispersed phase system can be a suspension comprising both dissolved peptide and dispersed peptide, the ratio of which is based on the solubility of the peptide in the final DP system. Alternatively, the resulting dispersed phase system can be an emulsion of two or more immiscible phases. In instances where peptide can precipitate out of solution during step (d), mixing or agitation or turbulence or energy by any suitable means can be used during the precipitation process in order to control or reduce particle size of the peptide during precipitation. Further, excipients such as salts, counter-ions, or solvents can be added to either or both the peptide solution from step (b) or the polymer solution of step (c) in order to facilitate re-precipitation to a particular solid-state form of the drug such as one or more salt forms of the peptide, solvate forms of the peptide, polymorphic forms of the peptide, and so on.

In one embodiment, the peptide solution from step (b) is sterile-filtered and the resulting filtered solution is added to the polymer solution of step (c).

In one embodiment, the addition of the peptide solution from step (b) to the polymer solution of step (c) is carried out with mixing or agitation to disperse the peptide solution into and throughout the polymer solution thereby forming the dispersed phase solution of step (d). In a further embodiment, mixing or agitation or energy can be used after the peptide solution of step (b) has been added to control particle size of any peptide that can precipitate out of solution during formation of the dispersed phase solution of step (d).

The dispersed phase comprises the peptide, the wall forming polymer excipient, propylene glycol, any excipients added to the solutions of step (b) or step (c), and the solvents or solvents used to form the solution in step (c).

Although the dispersed phase is typically hydrophobic, the dispersed phase can comprise an amount of water. This source of water can be from the use of water as a limited co-solvent in either step (b) or step (c). Alternatively, the source of water can be from the processing of the peptide, for example, bound water. Also, the source of water can be from any co-solvents, for example, the use of a hydroscopic solvent, or a solvent such as methanol or ethanol which may comprise a minor amount of water. In one embodiment, the amount of water that comprises the dispersed phase is less than about 5% by weight. In another embodiment, the amount of water that comprises the dispersed phase is less than about 1% by weight. In a further embodiment, the amount of water that comprises the dispersed phase is less than about 0.1% by weight. As such, the dispersed phase is substantially free of water when described as having any amount of water present that is in an amount less than about 5% by weight.

Step (e)

Step (e) relates to providing a continuous phase comprising water. The terms “continuous phase” and “continuous phase processing medium” are used synonymously throughout the specification to mean an aqueous phase that when contacted with the dispersed phase formed in step (d) causes an emulsion to form when the phases are combined under the conditions of thorough and/or sufficient mixing.

In one embodiment of the disclosed process, the continuous phase comprises greater than about 99.9% water. In another embodiment of the disclosed process, the continuous phase comprises greater than about 99% water. In a further embodiment of the disclosed process, the continuous phase comprises greater than about 95% water. In a yet further embodiment of the disclosed process, the continuous phase comprises greater than about 90% water. In still another embodiment of the disclosed process, the continuous phase comprises greater than about 80% water. In another embodiment, the continuous phase comprises greater than about 70% water.

In addition to water, the continuous phase or continuous phase processing medium can comprise one or more solvents chosen from a C₁-C₁₂ alcohol, inter alia, methanol, ethanol, n-propanol, iso-propanol, n-butanol, sec-butanol, tert-butanol, pentanol, hexanol, and benzyl alcohol; C₄-C₁₀ ether, inter alia, diethyl ether, diphenyl ether, methyl butyl ether, methyl tert-butyl ether, tetrahydrofuran, pyran, 1,2-dimethoxyethane (glyme), bis(2-methoxyethyl)ether (diglyme), and dioxane; C₃-C₁₂ ester, inter alia, methyl acetate, ethyl acetate, propyl acetate, methyl propionate, ethyl propionate, methyl lactate, and ethyl lactate; C₂-C₁₀ nitrile, inter alia, acetonitrile and propionitrile; C₃-C₁₂ ketone, inter alia, acetone, butanone, 2-pentanone, 3-pentanone, 2-hexanone, 3-hexanone, and acetophenone; substituted or unsubstituted benzene, inter alia, benzene, toluene, xylene (ortho, meta, para, or mixtures thereof), chlorobenzene, and nitrobenzene; C₅-C₂₀ hydrocarbon, inter alia, n-pentane, iso-pentane, n-hexane, n-octane, and iso-octane; C₁-Cl₂ haloalkane, inter alia, methylene chloride, chloroform, carbon tetrachloride, 1,2 dichloroethane, 1,1,1-trichloroethane, and 1,1,1,2-tetrafluroethane; C₂-C₁₂ nitroalkane, or other water soluble organic solvent, inter alia, dimethyl sulfoxide, dimethylformamide, diethylformamide, dimethylacetamide, and diethyl-acetamide.

Further, the continuous phase can comprise one or more water soluble processing aids such as a surfactant, emulsifier, or stabilizer. In addition, the continuous phase can comprise processing aids that assist in the extraction of one or more solvents, processing aids, or excipients from the dispersed phase. In particular, a preferred water-soluble continuous phase additive is poly(vinyl alcohol), PVA.

Step (f)

Step (f) relates to combining the dispersed phase and the continuous phase to form an emulsion. The liquid-liquid emulsion formed in step (f) comprises the dispersed phase which is discontinuous in the continuous phase processing medium. The emulsion can be formed by any variety of appropriate methods. One embodiment includes emulsification by static methods such as static mixers, diffuser plates, screen or membrane or diffuser gaskets, turbulent flow; another example includes emulsification using homogenizers, mixers, blenders, agitation, ultrasound or ultrasonic energy and the like. Another embodiment includes the use of nozzles or jets to create the emulsion comprising a discontinuous phase within the continuous phase liquid either alone or through the combined use of other techniques. A further embodiment can include processes that employ one or more such steps or methods during preparation of the emulsion.

The ratio of the dispersed phase mass to the continuous phase mass is from about 1:1.1 to about 1:200.

In one embodiment, the ratio of the dispersed phase mass to the continuous phase mass is from about 1:2 to about 1:50. In another embodiment, the ratio of the dispersed phase mass to the continuous phase mass is from about 1:2 to about 1:20. In a further embodiment, the ratio of the dispersed phase mass to the continuous phase mass is from about 1:2 to about 1:15. In a yet further embodiment, the ratio of the dispersed phase mass to the continuous phase mass is from about 1:2 to about 1:10. In another embodiment, the ratio of the dispersed phase mass to the continuous phase mass is from about 1:2 to about 1:5. However, the ratio of the dispersed phase mass to the continuous phase mass can have any value chosen by the formulator, for example, 1:1.1, 1:1.2, 1:1.3, 1:1.4, 1:1.5, 1:1.6, 1:1.7, 1:1.8, 1.1.9, 1:2, 1:2.1, 1:2.2, 1:2.3, 1:2.4, 1:2.5, 1:2.6, 1:2.7, 1:2.8, 2.2.9, 1:3, 1:3.1, 1:3.2, 1:3.3, 1:3.4, 1:3.5, 1:3.6, 1:3.7, 1:3.8, 3.3.9, 1:4, 1:4.1, 1:4.2, 1:4.3, 1:4.4, 1:4.5, 1:4.6, 1:4.7, 1:4.8, 4.4.9, and 1:5. Included herein are also any smaller fractional values, for example, 1:1.11, 1:1.25, 1:2.33, 1:2.47, 1:1.501, and 1:4.62.

Step (g)

Step (g) relates to combining the emulsion formed in step (f) with an extraction phase comprising water. In one embodiment, the extraction phase comprises greater than about 99.9% water. In another embodiment of the disclosed process, the extraction phase comprises greater than about 99% water. In a further embodiment of the disclosed process, the extraction phase comprises greater than about 97% water. In a yet further embodiment of the disclosed process, the extraction phase comprises greater than about 95% water. In another embodiment of the disclosed process, the extraction phase comprises greater than about 90% water. In still a further embodiment of the disclosed process, the extraction phase comprises greater than about 80% water. In another embodiment of the disclosed process, the extraction phase comprises greater than about 70% water.

Step (g) is conducted with any form of adequate mixing or turbulent flow that allows for the intimate and complete mixing of the emulsion formed in step (f) with the extraction phase provided herein.

Step (h)

Step (h) relates to forming microparticles. In one embodiment, step (g) and step (h) are combined into one continuous step; however, the formulator has the option to conduct solvent extraction to the desired degree of completion as can be determined by the total residual solvent content of the dry product.

Step (i)

The disclosed process can further comprise step (i) that encompasses isolation of the formed microparticles. As such, any process that the formulator can choose for isolating the microparticles is encompassed within the disclosed processes. Without being limiting, the formulator may choose to collect and isolate the microparticles by physically filtering the microparticles or the microparticles may be isolated by other suitable methods including, for example, spray drying, tangential filtration, centrifugation, evaporation, freeze drying, lyophilization, or by using combinations of two or more suitable methods.

The microparticles formed by the disclosed process can comprise a relatively narrow average particle diameter size distribution evidenced by a minimized percentage of relatively fine and/or relatively large microparticles. To this end, relative microparticle size distributions can be expressed by a particle size fraction. For example, the quantity d₅₀ is the mean microparticle size as measured in micrometers (μm); thus, d₅₀ is the microparticle diameter at which 50% of the particles have a smaller diameter and at which 50% have a larger diameter. The quantity d₉₀ is the diameter at which 90% of the microparticles comprise a diameter less than the value of d₉₀; thus, d₉₀ is also equal to the diameter at which 10% of the microparticles have a larger diameter. The quantity d₁₀ is the diameter at which 10% of the microparticles comprise a diameter less than the value of d₁₀; thus, d₁₀ is also equal to the diameter at which 90% of the microparticles have a larger diameter.

The microparticles formed by the disclosed process can have a mean particle size of from about 10 nm to about 2 mm. In one embodiment, the microparticles have a mean particle size of from about 20 μm to about 70 μm. In another embodiment, the microparticles have a mean particle size of from about 20 μm to about 50 μm. In a further embodiment, the microparticles have a d₁₀ particle size distribution of from about 1 ηm to about 20 μm. In a yet another embodiment, the microparticles have a d₁₀ particle size distribution of from about 3 μm to about 15 μm. In a yet further embodiment, the microparticles have a d₁₀ particle size distribution of from about 4 μm to about 12 μm. In a still another embodiment, the microparticles have a d₉₀ particle size distribution of from about 50 μm to about 100 μm. In a still further embodiment, the microparticles have a d₉₀ particle size distribution of from about 50 μm to about 80 μm. In a yet still another embodiment, the microparticles have a d₉₀ particle size distribution of from about 50 μm to about 70 μm. In a yet still further embodiment, the microparticles have a d₉₀ particle size distribution of from about 30 μm to about 60 μm.

The disclosed process further provides for an encapsulation efficiency of at least about 50%. In one embodiment, the encapsulation efficiency is from about 90% to about 99.5%. In another embodiment, the encapsulation efficiency is from about 60% to about 90%. In a further embodiment, the encapsulation efficiency is from about 70% to about 99%. In a yet further embodiment, the encapsulation efficiency is from about 95% to about 99.9%.

The term “encapsulation efficiency” as used herein means the percentage of peptide entrapped in the final microparticle product relative to the percentage of peptide added into the encapsulation process. For example, if a dispersed phase system in step (d) is prepared containing 25% by weight of drug (based on the total combined weight in the dispersed phase of drug and polymer and other excipients to be encapsulated) and if the final dry microparticle product is found to contain 19% by weight drug, then the encapsulation efficiency would be 76%.

Further aspects of the disclosed processes include the incorporation into the microparticles other excipients that can be beneficial for other clinical, diagnostic, surgical, or medical purposes, especially when the other excipient acts in concert with or synergistically with the peptide. Examples include agents that provide adjuvant properties, radio-opacity, radionucleotides, contrast agents, imaging agents, magnetic agents, and the like. Applications where these types of devices might be useful include any variety of medical imaging and diagnostics applications including, for example, MRI-based imaging such as metal oxide particles or iron oxide particles (including, for example super paramagnetic iron oxide, or SPIO, particles) and gadolinium-containing agents, among others. The microparticle compositions of the disclosed processes can also be prepared containing any of a variety of other dyes, contrast agents, fluorescent markers, imaging agents, magnetic agents, and radiologic agents used in any variety of medical diagnostic and imaging technologies.

### Compositions and Uses

Disclosed herein are microparticles made by the disclosed methods as well as methods for treating a human or an animal by administering the microparticles to a human or an animal in need of treatment. The microparticles disclosed herein have a slow and controlled release rate that can be adjusted by the formulator. Because many of the conditions treatable by active peptides, inter alia, goserelin (treatment of hormone-sensitive cancers, for example, breast cancer and prostate cancer), leuprolide (treatment of hormone-sensitive cancers, for example, breast cancer and prostate cancer, precocious puberty, control of ovarian stimulation in In Vitro Fertilization (IVF), and paraphilias), and octreotide (treatment for acromegaly, diarrhea and flushing episodes associated with carcinoid syndrome, and treatment of diarrhea in patients with vasoactive intestinal peptide-secreting tumors (VIPomas)). In addition, other peptides can be delivered that enhance one or more desirable biological response in a human. For example, oxytocin can be effectively delivered to a woman soon after birth to help stimulate the “let down reflex” for nursing mothers by causing lactation at the mammary glands, causing milk to be “let down” into a collecting chamber, from where it can be extracted by compressing the areola and sucking at the nipple.

As such, further disclosed herein are compositions useful for treating one or more diseases or conditions that can be effected by the delivery to a subject of one or more bioactive peptides. The compositions comprise microparticles that have an even sustained release profile instead of a sudden “burst” or rapid release of the peptide. For example, the disclosed microparticles can release a peptide at a slower rate than compared to a control, wherein the control is microparticle that has not been made using (and thus does not contain) propylene glycol. The microparticles disclosed herein comprise one or more peptides, a polymer excipient, and propylene glycol. The amount of propylene glycol can be at least about 0.01%, 0.1%, or 1% by weight of propylene glycol. In other examples, the amount of propylene glycol can be from about 0.05% to 15%, from about 0.1% to 10%, from about 1% to 10%, or from about 1% to 5% by weight of propylene glycol. In still further examples, the disclosed microparticles can have about 0.01%, 0.1%, 1%, 5%, 10%, 15%, 20%, 25%, 30%, 35%, 40%, 45%, 50%, 55%, 60%, 65%, 70%, 75%, 80%, 85%, or 90% by weight of propylene glycol, where any of the stated values can form an upper or lower endpoint of a range. In some aspects, the disclosed microparticles have a residual amount of propylene glycol.

The disclosed microparticles can have any of the peptides, polymer excipients, or excipients as disclosed hereinabove.

The present disclosure relates to a method of treating a human or a mammal comprising administering to a human or mammal in need of treatment, an effective amount of a microparticle comprising one or more bioactive peptides.

Further, the present disclosure relates to the use of a disclosed microparticle for the manufacture of a medicament.

The present disclosure also relates to a method for treating a hormone-sensitive cancer comprising administering to a patient having a hormone-sensitive cancer, an effective amount of a microparticle comprising a bioactive peptide useful in treating a hormone-sensitive cancer.

### EXAMPLES

The following examples are set forth below to illustrate the methods and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

Efforts have been made to ensure accuracy with respect to numbers (e.g., amounts, temperature, pH, etc.) but some errors and deviations should be accounted for. Unless indicated otherwise, parts are parts by weight, temperature is in ° C. or is at ambient temperature, and pressure is at or near atmospheric. There are numerous variations and combinations of conditions, e.g., component concentrations, temperatures, pressures, and other reaction ranges and conditions that can be used to optimize the product purity and yield obtained from the described process. Only reasonable and routine experimentation will be required to optimize such process conditions.

### Example 1

A 20 weight percent polymer solution was prepared by dissolving 1.8 g of 50:50 poly(DL-lactide-co-glycolide) (“DL-PLG”) in 7.2 g of dichloromethane. (The DL-PLG has an inherent viscosity of 0.31 dL/g.) In a separate flask, goserelin (5-oxo-prolylhistidyl-tryptophylseryltyrosyl(O-tert butyl)serylvalylarginylprolylNNHC(O)NH₂) (200 mg) was dissolved in propylene glycol (2 mL). The two solutions were combined with homogenation using a Polytron probe mixer and then injected using a 10 mL syringe into a 250 mL beaker containing 150 g of 2 wt % poly (vinyl alcohol) and 2.4 grams of methylene chloride stirred at 1000 rpm with a Silverson L4R-TA probe mixer with high shear screen. The resulting emulsion was then poured into 3 L of water at 25° C. and stirred until microparticles are formed. After 60 minutes the microparticles were collected by passing between 125 and 25 micrometer test sieves. The microparticles collected on the 25 micrometer test sieve were rinsed with 2 L of de-ionized water then air dried. Air drying was conducted by placing the 25 micrometer sieve in a laminar flow hood for 48 hours to allow the product to dry by evaporation. After drying, the microparticles were transferred to a scintillation vial. The resulting microparticles have an encapsulation efficiency of 70%.

### Example 2 (Comparative)

A 20 weight percent polymer solution was prepared by dissolving 1.8 g of 50:50 poly(DL-lactide-co-glycolide) (“DL-PLG”) in 7.2 g of dichloromethane. (The DL-PLG had an inherent viscosity of 0.31 dL/g.) Goserelin (5-oxo-prolylhistidyl-tryptophylseryltyrosyl(O-tert-butyl)serylvalylarginylprolylNHNHC(O)NH₂) (200 mg) was mixed with the polymer solution and the resulting mixture was homogenized using a Polytron probe mixer and then injected using a 10 mL syringe into a 250 mL beaker containing 150 g of 2 wt % poly(vinyl alcohol) and 2.4 grams of methylene chloride stirred at 1000 rpm with a Silverson L4R-TA probe mixer with high shear screen. The resulting emulsion was then poured into 3 L of water at 25° C. and stirred until microparticles were formed. After 60 minutes the microparticles were collected by passing between 125 and 25 micrometer test sieves. The microparticles collected on the 25 micrometer test sieve were rinsed with 2 L of de-ionized water then air dried. Air drying was conducted by placing the 25 micrometer sieve in a laminar flow hood for 48 hours to allow the product to dry by evaporation. After drying, the microparticles were transferred to a scintillation vial. The resulting microparticles have an encapsulation efficiency of 34%.

Table I provides the in vitro release time intervals of goserelin into phosphate buffered saline (PBS) for the microparticles of Example 1 and 2.

TABLE I
Cumulative percent goserelin released (%) at various
times after exposure to PBS.

| Example | 1 day | 2 days | 3 days | 6 days | 7 days |
|---|---|---|---|---|---|
| 1 | 3.21 | 3.42 | 3.60 | 5.40 | 6.91 |
| 2 | 31.96 | 33.71 | 34.32 | 38.24 | 38.86 |

### Example 3

Solubilities at various concentration ranges were estimated based on visual observations of solutions prepared at the indicated peptide concentration levels listed in Table II.

TABLE II
Peptide solubilities in polyols including propylene glycol,
glycerol, and poly(ethylene glycol) (PEG-400).

| | | Peptide solubility at | | |
|---|---|---|---|---|
| | | specified concentration level | | |
| | | | At 10 | |
| Peptide | Solvent | <10 mg/mL | mg/mL | 100 mg/mL |
| Goserelin | Propylene glycol | Soluble | Soluble | Soluble |
| | Glycerol | (solubility | Not soluble | Not soluble |
| | | less than | | |
| | | 10 mg/mL) | | |
| | PEG-400 | Not soluble | Not soluble | Not soluble |
| Octreotide | Propylene glycol | Soluble | Soluble | Soluble |
| Leuprolide | Propylene glycol | Soluble | Soluble | soluble |

While particular embodiments of the present disclosure have been illustrated and described, it would be obvious to those skilled in the art that various other changes and modifications can be made without departing from the spirit and scope of the disclosure. It is therefore intended to cover in the appended claims all such changes and modifications that are within the scope of this disclosure.

What is claimed is:

1. A process for preparing peptide-containing microparticles, comprising:
a. providing one or more peptides
b. dissolving the one or more peptides in a solution comprising propylene glycol to form a peptide solution;
c. providing a solution comprising a polymer excipient dissolved or dispersed therein;
d. combining the peptide solution from (b) with the polymer excipient solution from (c) to form a dispersed phase;
e. providing a continuous phase comprising water;
f. combining the dispersed phase and the continuous phase to form an emulsion;
g. combining the emulsion formed in step (f) with an extraction phase comprising water; and
h. forming microparticles.

2. The process according to claim 1, wherein the peptide is a naturally-occurring bioactive peptide or a synthetic bioactive peptide having solubility in propylene glycol in an amount of at least 1 mg/mL.

3. The process according to claim 1, wherein the peptide comprises glucagon-like peptide, adrenocorticotropic hormone, an opioid peptide, a neuroactive peptide, a growth factor, a growth hormone regulatory peptide, a hormone-regulating peptide, a metabolic peptide, or an LHRH analog.

4. The process according to claim 1, wherein the peptide is a octreotide, goserelin, leuprolide, somatostatin, a somatostatin analog, a GLP-1 peptide analog, a GLP-2 peptide analog, a PYY peptide, oxytocin, angiotensin, bradykinin, or arginine vasopressin.

5. The process according to claim 1, wherein the peptide solution of step (b) is a homogeneous solution.

6. The process according to claim 1, wherein the peptide solution of step (b) is a suspension comprising dissolved peptide and suspended peptide.

7. The process according to claim 1, wherein the peptide solution of step (b) further comprises one or more organic solvents.

8. The process according to claim 7, wherein the one or more organic solvent comprises a C₁-C₁₂ alcohol, C₄-C₁₀ ether, C₃-C₁₂ ester, C₂-C₁₀ nitrile, C₃-C₁₂ ketone, substituted or unsubstituted benzene, C₅-C₂₀ hydrocarbon, C₁-C₁₂ haloalkane, C₂-C₁₂ nitroalkane, or other water soluble organic solvent.

9. The process according to claim 1, wherein the peptide solution of step (b) further comprises water.

10. The process according to claim 1, wherein the solution from step (c) comprises an organic solvent.

11. The process according to claim 10, wherein the one or more organic solvent comprises a C₁-C₁₂ alcohol, C₂-C₁₀ ether, C₃-C₁₂ ester, C₂-C₁₀ nitrile, C₃-C₁₂ ketone, substituted or unsubstituted benzene, C₅-C₂₀ hydrocarbon, C₁-C₁₂ haloalkane, or C₂-C₁₂ nitroalkane.

12. The process according to claim 1, wherein the polymer excipient in step (c) is a homopolymer, copolymer, or block copolymer comprising:
a. polyesters;
b. polyanhydrides;
c. polyorthoesters;
d. polyphosphazenes;
e. polyphosphates;
f. polyphosphoesters;
g. polydioxanones;
h. polyphosphonates;
i. polyhydroxyalkanoates;
j. polycarbonates;
k. polyalkylcarbonates;
l. polyorthocarbonates;
m. polyesteramides;
n. polyamides;
o. polyamines;
p. polypeptides;
q. polyurethanes;
r. polyetheresters;
s. polyalkylene glycols;
t. polyalkylene oxides;
u. polysaccharides;
v. polyvinyl pyrrolidones; or
w. combinations or blends thereof.

13. The process according to claim 1, wherein the polymer excipient in step (c) comprises:
i) poly(lactide)-co-(polyalkylene oxide);
ii) poly(lactide-co-glycolide)-co-(polyalkylene oxide);
iii) poly(lactide-co-caprolactone)-b-(polyalkylene oxide); or
iv) poly(lactide-co-glycolide-co-caprolactone)-b-(polyalkylene oxide).

14. The process according to claim 1, wherein the polymer excipient in step (c) comprises:
i) poly(lactide)-co-poly(vinylpyrrolidone);
ii) poly(lactide-co-glycolide)-co-poly(vinylpyrrolidone);
iii) poly(lactide-co-caprolactone)-b-poly(vinylpyrrolidone); or
iv) poly(lactide-co-glycolide-co-caprolactone)-b-poly(vinylpyrrolidone).

15. The process according to claim 1, wherein the polymer excipient in step (c) comprises:
i) poly(lactide);
ii) poly(glycolide);
iii) poly(caprolactone);
iv) poly(valerolactone);
v) poly(hydroxybutyrate);
vi) poly(lactide-co-glycolide);
vii) poly(lactide-co-caprolactone);
viii) poly(lactide-co-valerolactone);
ix) poly(glycolide-co-caprolactone);
x) poly(glycolide-co-valerolactone);
xi) poly(lactide-co-glycolide-co-caprolactone); or
xii) poly(lactide-co-glycolide-co-valerolactone).

16. The process according to claim 1, wherein the polymer excipient in step (c) is poly(lactide).

17. The process according to claim 1, wherein the polymer excipient in step (c) is poly(lactide-co-glycolide).

18. The process according to claim 1, wherein the microparticles comprise from about 1% to about 10% by weight of peptide.

19. The process according to claim 1, further comprising isolating the microparticles by centrifugation or filtration.

20. The process according to claim 1, wherein the microparticles have a mean particle size of from about 40 μm to about 90 μm.

21. The process according to claim 1, wherein the microparticles have a d₁₀ particle size distribution of from about 4 μm to about 12 μm.

22. The process according to claim 1, wherein the microparticles have a d₉₀ particle size distribution of from about 90 μm to about 110 μm.

23. The process according to claim 1, wherein the peptide solution of step (b) comprises from about 10% to 90% by weight of propylene glycol.

24. The process according to claim 1, wherein the solution of step (c) comprises ethyl acetate or methylene chloride.

25. A microparticle, comprising: a bioactive peptide, a polymer excipient, and propylene glycol.

26. The microparticle of claim 25, wherein the peptide is a naturally-occurring bioactive peptide or a synthetic bioactive peptide having solubility in propylene glycol in an amount of at least 1 mg/mL.

27. The microparticle of claim 25, wherein the peptide comprises glucagon-like peptide, adrenocorticotropic hormone, an opioid peptide, a neuroactive peptide, a growth factor, a growth hormone regulatory peptide, a hormone-regulating peptide, a metabolic peptide, or an LHRH analog.

28. The microparticle of claim 25, wherein the peptide is a octreotide, goserelin, leuprolide, somatostatin, a somatostatin analog, a GLP-1 peptide analog, a GLP-2 peptide analog, a PYY peptide, oxytocin, angiotensin, bradykinin, or arginine vasopressin.

29. The microparticle of claim 25, wherein the polymer excipient is a homopolymer, copolymer, or block copolymer comprising:
a. polyesters;
b. polyanhydrides;
c. polyorthoesters;
d. polyphosphazenes;
e. polyphosphates;
f. polyphosphoesters;
g. polydioxanones;
h. polyphosphonates;
i. polyhydroxyalkanoates;
j. polycarbonates;
k. polyalkylcarbonates;
l. polyorthocarbonates;
m. polyesteramides;
n. polyamides;
o. polyamines;
p. polypeptides;
q. polyurethanes;
r. polyetheresters;
s. polyalkylene glycols;
t. polyalkylene oxides;
u. polysaccharides;
v. polyvinyl pyrrolidones; or
w. combinations or blends thereof.

30. The microparticle of claim 25, wherein the polymer excipient comprises:
i) poly(lactide)-co-(polyalkylene oxide);
ii) poly(lactide-co-glycolide)-co-(polyalkylene oxide);
iii) poly(lactide-co-caprolactone)-b-(polyalkylene oxide); or
iv) poly(lactide-co-glycolide-co-caprolactone)-b-(polyalkylene oxide).

31. The microparticle of claim 25, wherein the polymer excipient comprises:
i) poly(lactide)-co-poly(vinylpyrrolidone);
ii) poly(lactide-co-glycolide)-co-poly(vinylpyrrolidone);
iii) poly(lactide-co-caprolactone)-b-poly(vinylpyrrolidone); or
iv) poly(lactide-co-glycolide-co-caprolactone)-b-poly(vinylpyrrolidone).

32. The microparticle of claim 25, wherein the polymer excipient comprises:
i) poly(lactide);
ii) poly(glycolide);
iii) poly(caprolactone);
iv) poly(valerolactone);
v) poly(hydroxybutyrate);
vi) poly(lactide-co-glycolide);
vii) poly(lactide-co-caprolactone);
viii) poly(lactide-co-valerolactone);
ix) poly(glycolide-co-caprolactone);
x) poly(glycolide-co-valerolactone);
xi) poly(lactide-co-glycolide-co-caprolactone); or
xii) poly(lactide-co-glycolide-co-valerolactone).

33. The microparticle of claim 25, wherein the polymer excipient is poly(lactide).

34. The microparticle of claim 25, wherein the polymer excipient is poly(lactide-co-glycolide).

35. The microparticle of claim 25, wherein the microparticle comprises from about 1% to about 10% by weight of peptide.

36. The microparticle of claim 25, wherein the microparticle releases the bioactive peptide into phosphate buffered saline at a rate less than that from a control microparticle without propylene glycol.

37. The microparticle of claim 25, wherein the microparticles have a mean particle size of from about 40 μm to about 90 μm.

38. The microparticle of claim 25, wherein the microparticles have a d₁₀ particle size distribution of from about 4 μm to about 12 μm.

39. The microparticle of claim 25, wherein the microparticles have a d₉₀ particle size distribution of from about 90 μm to about 110 μm.

40. The microparticle of claim 25, wherein microparticle comprises at least about 0.01% by weight of propylene glycol.
